(19) United States
(12) Patent Application Publication
(10) Pub. No.: US 2007/0270672 A1
(43) Pub. Date: Nov. 22, 2007

(54) Wearable Sensor Device and System

(76) Inventor: Paul G. Hayter, Mountain View, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/574,346

(22) PCT Filed: Aug. 31, 2005
(86) PCT No.: PCT/US05/31271

§ 371 Date: Aug. 1, 2007

Related U.S. Application Data
(60) Provisional application No. 60/606,334, filed on Aug. 31, 2004.

(51) Int. Cl. A61B 5/00

(52) U.S. Cl. 600/309

(57) ABSTRACT

A system for determining the level of an analyte in a physiological fluid of a live individual is described. A wearable sensor periodically obtains data representative of the level of the analyte and has a passive RFID tag that stores the data. A receiver wirelessly interrogates the sensor with an RF interrogation signal. The RFID tag modulates or otherwise modifies the wireless interrogation signal using the data and the receiver receives back the modulated or otherwise modified interrogation signal and extracts the data from it. The receiver then determines, from the data, the level of the analyte in the fluid. The sensor may be a photometric or colorimetric sensor or an electrochemical sensor. The receiver may be, or be incorporated into, a hand-held device, a portable device, a PDA, a mobile telephone, or a laptop computer. The resulting system is more versatile and consumes less power than conventional systems.

### PRIORITY

This application claims priority benefits under 35 U.S.C. §§ 120 and 371 of International Application PCT/US2005/031271 filed on 31 Aug. 2005, which claims priority benefits to U.S. Provisional Application Ser. No. 60/606,334 filed on 31 Aug. 2004, which both applications are hereby incorporated by reference in their entireties into this application.

### FIELD OF THE INVENTION

The invention relates to a continuous sensor for use, in healthcare management, law-enforcement, dope-testing, sanitation or otherwise, for measuring the concentration of any analyte, such as glucose, lactate, urate, alcohol, therapeutic drugs, recreational drugs, performance-enhancing drugs, biomarkers indicative of diseased conditions, hormones, antibodies, metabolites of any of the aforesaid, combinations of any of the aforesaid, other similar indicators or any other analyte in a fluid, especially a physiological fluid such as blood, interstitial fluid (ISF) or urine. Much of the following discussion will concentrate upon the use of such a sensor for the purpose of blood glucose measurement and control but the principles discussed are much more widely applicable; indeed, they are applicable to the detection of any analyte in any fluid.

### BACKGROUND TO THE INVENTION

Glucose monitoring is a fact of everyday life for diabetic individuals. The accuracy of such monitoring may have significant impact on the quality of life. Generally, a diabetic patient measures blood glucose levels several times a day to monitor and control blood sugar levels. Failure to control blood glucose levels within a recommended range can result in serious healthcare complications such as limb amputation and blindness. Furthermore, failure to accurately measure blood glucose levels may result in hypoglycaemia. Under such conditions the diabetic patient may initially enter a comatose state, and if untreated may die. Therefore, it is important that accurate and regular measurements of blood glucose levels are performed.

People suffering from diabetes are often at a higher risk of other diseases. Diabetes also contributes to kidney disease, which occurs when the kidneys do not filter properly and protein leaks into urine in excessive amounts, which eventually can cause kidney failure. Diabetes is a cause of damage to the retina at the back of the eye and also increases risk of cataracts and glaucoma. Nerve damage caused by diabetes may interfere with the ability to sense pain and contributes to serious infections. A number of glucose meters are currently available which permit a user to test the glucose level in a small sample of body fluid.

Many of the glucose meter designs currently available make use of a disposable test sensor, e.g., a strip, which in combination with the meter, electrochemically or photometrically measures the amount of glucose in the blood sample. To use these meters, the user first punctures a finger or other body part using a lancet to produce a small sample of blood or interstitial fluid. The sample is then transferred to a disposable test strip. The test strips are typically held in packaging containers or vials prior to use. Generally, test strips are quite small and the sample receiving area is even smaller. Usually, the disposable strip is inserted into a meter through a port in the meter housing prior to performing a test for an analyte in body fluids such as blood, ISF or urine etc.

Other meter designs are capable of providing more or less continuous measurements. One example is described in U.S. patent application Ser. No. 10/882,994, the entire contents of which are herein incorporated by reference. In this example, the system extracts interstitial fluid samples and monitors the level of glucose contained within it. The components of the system are a disposable cartridge, a local controller module, and a remote controller module. The disposable cartridge includes a sampling module that extracts the interstitial fluid sample from the skin and an analysis module that measures the glucose level. Examples of suitable sampling and analysis modules are described in International Patent Application WO 02/49507, the entire content of which is herein incorporated by reference. In particular, the system of U.S. patent application Ser. No. 10/882,994 may use that multi-use electrochemical or photometric analyte sensors discussed in WO 02/49507. A characteristic of the system described in U.S. patent application Ser. No. 10/882,994 is that the sampling and analysis modules are designed to be worn on the body for a relatively short period of time, say 12 hours, after which they are disposed of. Each measurement of glucose level is transmitted via an RF link from a local controller module that is attached to the sampling an analysis modules, to a remote controller module. Because the local controller module is to be worn for 12 hours at a time, it must be relatively lightweight and relatively unsophisticated; most of the detailed analysis of the glucose measurements takes place only in the remote control module.

Another example of a meter design capable of providing more or less continuous measurements is described in U.S. patent application Ser. No. 11/200,768, the entire contents of which are herein incorporated by reference. A fluorescent light-emitting bead is implanted just beneath the skin. The bead includes a fluorescent reagent that emits fluorescent light as a result of absorbing incident light, the characteristics of the emitted fluorescent light being dependent on the concentration of glucose that is in contact with the bead. Fluorescent reactants that can be included in such a fluorescent light-emitting bead, and their behaviour when in communication with an analyte, are described in U.S. Pat. Nos. 5,342,789, 6,040,194, and 6,232,130, the entire contents of which are herein incorporated by reference. The bead can also include an encapsulating material such as, for example, alginate. Any envelope that is substantially impermeable to the reagent, but is permeable to the analyte is suitable. An adhesive fluorescence measurement patch is adhered to the skin over the bead and communicates with a remote module via an RF link to transmit each glucose measurement. Again, the patch is relatively unsophisticated and most of the detailed analysis of the glucose measurements takes place only in the remote control module.

To enable the local module or skin patch as the case may be to take glucose measurements and to communicate the glucose measurement data each time to the remote module, the local module or skin patch must possess a source of power. Typically, this would be a battery. Transferring the data to the remote module typically consumes the greater part of the power it is able to supply, which means that the battery life is constrained for the most part by the need to power the RF communication. This is a particular problem because the local device or skin patch are for the most part out of sight and a low battery level may not immediately be apparent to the user. The result can be false measurements, or failure to supply measurement data to the remote module, either of which can seriously compromise the welfare of the user, eventually leading in the worst cases to coma and death.

Similar problems arise with the remote module too. If the batteries in the remote module run low, exactly the same result may ensue.

### SUMMARY OF THE INVENTION

The present invention is designed to address the problems outlined above. Our solution is to propose a change in the way that the wireless link between the local module or patch and the remote module is used. In particular, we propose not to insist that every measurement of glucose or other analyte be transmitted to the remote module when it is taken. Instead, we disclose that the timing of the transmission of the data to the remote module be under the control of the remote module. By doing so, the RF receiver circuits of the remote module need not be active at all times, just in case a signal is received from the local module or patch. Instead, those circuits can be quiescent or powered down completely until the remote module determines, according to its schedule, that the transmission of data is required.

Whilst those problems have been described particularly with reference to the management of diabetes, where accurate and timely measurement is absolutely essential, we nonetheless regard the problem as more general. Indeed, if one is testing any physiological fluid using a sensor that is to be exposed to the fluid, and a receiver with which it wirelessly communicates, and one wishes to avoid the inconvenience of frequent battery changes and the possibility of false readings, the present invention will be of considerable assistance. Therefore, one embodiment of the present invention is that it involves a system for determining the level of an analyte in a physiological fluid of a live individual, comprising:

a wearable sensor that is adapted to obtain periodically, data representative of the level of the analyte, and has a wireless device adapted to convey the data wirelessly when wirelessly interrogated; and

a receiver adapted to operate as follows:

to wirelessly interrogate the sensor; and

to receive the data conveyed by the wireless device.

Depending on the level of sophistication of the wearable sensor, it may itself determine the level of the analyte, for example by converting a current measurement into a glucose concentration measurement, in which case the data will directly represent the level of the analyte in the fluid. Alternatively, it may indirectly represent the level of the analyte in the fluid, with the receiver being adapted to determine, from the data, the level of the analyte in the fluid. The present invention finds application in integrated systems for measuring and treating medical disorders or diseased conditions. Thus, the level of the analyte may be diagnostic of a medical disorder or diseased condition, such as diabetes. To complete the integrated system, when the medical disorder or diseased condition is remediable by the administration of a drug, the system may further include a drug dispensing unit for dispensing the drug. The drug dispensing unit is preferably adapted to dispense the drug in an amount that depends upon the level of the analyte in the fluid as conveyed by the sensor or determined by the receiver.

Analytes for which the system may test include glucose, HbA1C, lactate, cholesterol, alcohol, a ketone, urate, a therapeutic drug, a recreational drug, a performance-enhancing drug, a biomarker indicative of a diseased condition, a hormone, an antibody, a metabolite of any of the aforesaid, a combination of any of the aforesaid, or another similar indicator.

In an integrated system, where the analyte is glucose, the drug should be one that promotes cellular uptake of glucose, such as a drug comprising insulin or an insulin analog. The dispensing unit may comprise an infusion pump or other mechanism, preferably a wearable pump or mechanism, adapted to dispense the drug directly into the body of the user concerned.

As discussed, the receiver may be, or be incorporated within, a local device worn by the user concerned or a device remote from the user concerned. Alternatively, there may be a local device and a device remote from the user concerned with the local and remote devices in wireless communication with one another and adapted to transfer from the local device to the remote device either the data received from the sensor or the level of the analyte as determined by the receiver or both. Remote devices may be used as parental monitors for those suffering from childhood diabetes.

If the remote device is able to establish a communications link with a public switched telephone network or another circuit-switched communications network, or a mobile telephony network, the internet or another packet-switched communications network or the local device is able to establish a communications link with a mobile telephony network or another wireless communications network, either may be used to inform a physician or care-giver of a subject's state of health or notify the emergency services of the onset of an acute event.

In the former case, the communications link would typically be used to transmit the data received from the sensor or the level of the analyte as determined by the receiver, information concerning the variation of either over time, or other similar information.

In either case, it may be used to transmit an alarm condition such as an abnormal analyte level, an abnormal analyte level for a certain time of day, an abnormal analyte level as compared with dietary intake, abnormal or non-functional wireless transfer of information from the sensor or the local device, abnormal physiological fluid sampling frequency, abnormal establishment, or non-establishment, of wireless communication from the sensor or the local device, abnormal storage of information in the sensor or other alarm conditions.

Having explained that causing the receiver to determine the schedule of wireless transmission from the wearable sensor reduces the power consumption of the overall system, we now explain some further adaptations that either contribute to this aim or contribute to the aim of keeping the wearable sensor as small and lightweight as possible.

The first adaptation is for the wearable sensor, instead of using its own power supply to transmit data to the receiver, to use the power supply of the remote device. This can happen as follows. The receiver interrogates the sensor by issuing a wireless interrogation signal, and the sensor extracts energy from the wireless interrogation signal and uses the energy extracted to transmit data wirelessly to the receiver. Devices that operate in this way are known.

An alternative is for the wearable sensor not to transmit data in the conventional sense at all. The receiver will still interrogate the sensor by issuing a wireless interrogation signal, but in this case the sensor modulates or otherwise modifies the wireless interrogation signal using the data. The receiver receives back the modulated or otherwise modified interrogation signal and extracts the data from it. One way of achieving this mode of operation is to use a wireless device that back-scatters the interrogation signal, such as an RFID tag.

A second adaptation takes the first of these ideas even further. The wearable sensor, instead of using its own power supply to obtain the data to be conveyed, for example by sampling the physiological fluid, again uses the power supply of the remote device. In this case, the receiver issues a wireless test signal and the wireless device extracts energy from the wireless test signal and uses the energy extracted to obtain the data to be conveyed.

So far, we have described the system, but the present invention also extends to the sensor. Thus, another statement of the present invention is that it involves a wearable sensor for use in determining the level of an analyte in a physiological fluid of a live user, the sensor being adapted to obtain periodically data representative of the level of the analyte, and having a wireless device adapted to convey the data wirelessly when wirelessly interrogated.

Preferred sensors are of the type that, when exposed to the physiological fluid, develops a measurable characteristic that is a function of the level of the analyte in the fluid and of a calibration quantity of the sensor, in which case the wireless device should hold and convey information representing the calibration quantity of the sensor. The advantage of this is that, returning to the system, the receiver can also receive the information representing the calibration quantity of the sensor and to use it when determining the level of the analyte in the fluid.

The term “calibration quantity” will now be explained. Variations in the manufacturing process result in sensors having different physical, chemical or other inherent properties that affect the way they respond to an analyte. Thus, different sensors will respond slightly differently to the same concentration of analyte in a fluid. Because they respond differently, their response must then be adjusted by an amount that is determined by calibration. The calibration process allows one to determine one or more adjustment coefficients that, when applied to the response of the sensor, will normalize it to a predefined standard. To help us to refer to the physical, chemical or other inherent characteristics of the sensor, we have coined the expression “calibration quantity”. A calibration quantity is some property that the sensor possesses that affects its response. It may be a single property, such as sensitivity; it may be a combination of many, such as sensitivity, non-linearity, hysteresis, etc. It may be some structural property such as size that contributes to its response behaviour, either by affecting other calibration quantities like sensitivity, or by making an individual contribution. All of these things, alone or together, are calibration quantities, from which it can be seen that the term denotes a broad class. It is to be distinguished from the one or more adjustment coefficients that are derived from the calibration process and, when applied to the response of the strip, will normalize it to a predefined standard. These coefficients are shorthand representations of calibration quantities; they are information representing the calibration quantities, but they are not the calibration quantities themselves, which are real properties of the sensors. Thus, where we wish to refer to the adjustment coefficients or any other information representing them, and therefore representing the calibration quantities of the sensors, for example a code pointing to a location in a look-up table at which the relevant adjustment coefficients may be found, we use the expression “information representing the calibration quantity.” The distinction is a simple one, but it is worth setting out here for the avoidance of doubt.

A particularly preferred form of sensor is the optometric sensor that described in U.S. patent application Ser. No. 11/200,768. Such a sensor comprises an intracorporeal part that is exposed to the physiological fluid by implantation in the user concerned and, when so exposed, develops a measurable characteristic, being an indicator of the extent to which exposure of the sensor to the fluid affects its optical characteristics, that is a function of the level of the analyte in the fluid, and an extracorporeal part that acquires the measurable characteristic of the intracorporeal part by transdermal wireless communication. In the present invention, it is the extracorporeal part that includes the wireless device.

As described in U.S. patent application Ser. No. 11/200,768, the transdermal wireless communication is transdermal optical transmission, the intracorporeal part comprises a fluorescent reagent that reversibly binds to the analyte. The measurable characteristic may be: a fluorescence intensity; an emission or excitation spectrum, peak, gradient or ratio; any one or more parts of such a spectrum; an emission polarization; an excited state lifetime; a quenching of fluorescence; a change over time of any of the aforesaid; any combination of the aforesaid; or any other indicator of the extent to which exposure of the fluorescent reagent to the fluid affects its fluorescence characteristics.

Preferred embodiments use a reagent comprising or labelled with a donor molecule and an acceptor molecule, where the measurable characteristic is an indicator of the extent to which non-radiative fluorescence resonance energy transfer occurs between the donor and the acceptor upon reversible binding of the reagent to the analyte. The reagent may comprise a specific binding pair, one of which is, or is labelled with, the donor molecule and the other of which is, or is labelled with, the acceptor molecule. The sensor may comprise an envelope that contains and is substantially impermeable to the reagent, but is permeable to the analyte. The envelope may be a microdialysis vessel or a microcapsule or an alginate bead optionally covered with a polylysine covering.

Other optometric sensors are for extracorporeal use and include means for extracting the physiological fluid, a reagent, and means for exposing the reagent to the fluid. The reagent may include a catalyst and a dye or dye precursor and the catalyst catalyses, in the presence of the analyte, the denaturing of the dye or the conversion of the dye precursor into a dye. For glucose analysis, the catalyst may be a combination of glucose oxidase and horseradish peroxidase, with the reagent including a leuco-dye. Suitable Leuco-dyes are 2,2-azino-di-[3-ethylbenzthiazoline-sulfonate], tetramethylbenzidine-hydrochloride and 3-methyl-2-benzothiazoline-hydrazone in conjunction with 3-dimethylamino-benzoicacide.

In such photometric or colorimetric sensors, the measurable characteristic may be: an opacity; a transparency; a fluorescence intensity; a transmissivity, a reflectivity, an absorptivity or an emissivity; a transmission, reflection, absorption, emission or excitation spectrum, peak, gradient or ratio; any one of more parts of such a spectrum; a colour; an emission polarization; an excited state lifetime; a quenching of fluorescence; a change over time of any of the aforesaid; any combination of the aforesaid; or any other indicator of the extent to which exposure of the sensor to the fluid affects its optical characteristics.

Extracorporeal electrochemical sensors comprising electrodes, means for extracting the physiological fluid, a reagent, and means for exposing the reagent to the fluid, may be used with this invention too. In such cases, the measurable characteristic may be: an inter-electrode impedance; an inter-electrode current; a potential difference; an amount of charge; a change over time of any of the aforesaid; any combination of the aforesaid; or any other indicator of the amount of electricity passing from one electrode to another, or the extent to which exposure of the sensor to the fluid generates electrical energy or electrical charge or otherwise affects the electrical characteristics of the sensor.

Such sensors may include a substrate, an electrode layer containing the electrodes, and at least a first reagent layer. For glucose analysis, the reagent layer may comprise glucose oxidase.

As discussed, the receiver may be, or may be incorporated into, a hand-held device, a portable device, a PDA, a mobile telephone, or a laptop computer. So may the remote device.

A suitable wireless device is an RFID tag, for example ISO 14443 or ISO 15693, 13.56 MHz or 2.45 GHz.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements), of which:

FIG. 1 shows a schematic plan view of a single use test strip for receiving a patient's blood, having an RFID tag integrated thereon. This figure is presented for purposes of illustration of some of the principles underlying the present invention.

FIG. 2 shows a schematic plan view of a single use test strip for receiving a patient's blood and a blood glucose meter, according to a further exemplary embodiment of the invention having an RFID tag integrated on the single use test strip having conductive tracks feeding to an edge of the test strip. This figure is also presented for purposes of illustration of some of the principles underlying the present invention.

FIG. 3 shows a schematic plan view of a single use test strip for receiving a patient's blood and a blood glucose meter, according to a further exemplary embodiment of the invention having an RFID tag integrated on the single use test strip. The RFID tag is written to by RF techniques during the manufacturing stage of the single use test strip Again, this figure is presented for purposes of illustration of some of the principles underlying the present invention.

FIG. 4 shows a schematic plan view of a multi use test strip or module in the form of a disc for receiving a patient's blood, having an RFID integrated thereon.

FIG. 5 shows a schematic plan view of a multi use test strip formed as an array. Each strip contained in the array has an RFID tag contained within it. Alternatively, or in addition a separate RFID tag can be used as the sole RFID tag. The RFID tag contains calibration code data specific to that multi use test strip 2.

FIG. 6 shows a system diagram depicting a system for extracting and monitoring a bodily fluid sample within which, for example, the embodiments of FIG. 4 or FIG. 5 can be used.

FIG. 7 shows a table of information which may be loaded from a RFID tag to the meter and from the meter to the RFID tag in accordance with example embodiments of the present invention.

FIG. 8 is a simplified block diagram depicting a system for extracting a bodily fluid sample and monitoring an analyte.

FIG. 9 is a simplified schematic diagram of an ISF sampling module being applied to a user's skin layer, with the dashed arrow indicating a mechanical interaction and the solid arrows indicating ISF flow or, when associated with element 28, the application of pressure.

FIG. 10 is a simplified block diagram of an analysis module, local controller module and remote controller module.

FIG. 11 is a simplified schematic illustration depicting interaction between a fluorescent light-emitting bead, light emitter and light detector.

FIG. 12 is a simplified schematic illustration depicting interaction between a fluorescent light-emitting bead implanted in a user's body, a light emitter, and a light detector for detecting fluorescent light that is relevant to various embodiments of the present invention.

FIG. 13A is a simplified cross-sectional view of an adhesive fluorescence measurement patch adhered to a user's body.

FIG. 13B is a simplified schematic depicting the operative interaction of various electrical and optical components, including a light emitter and a light detector, suitable for use in the adhesive fluorescence measurement patch of FIG. 13A.

### DETAILED DESCRIPTION

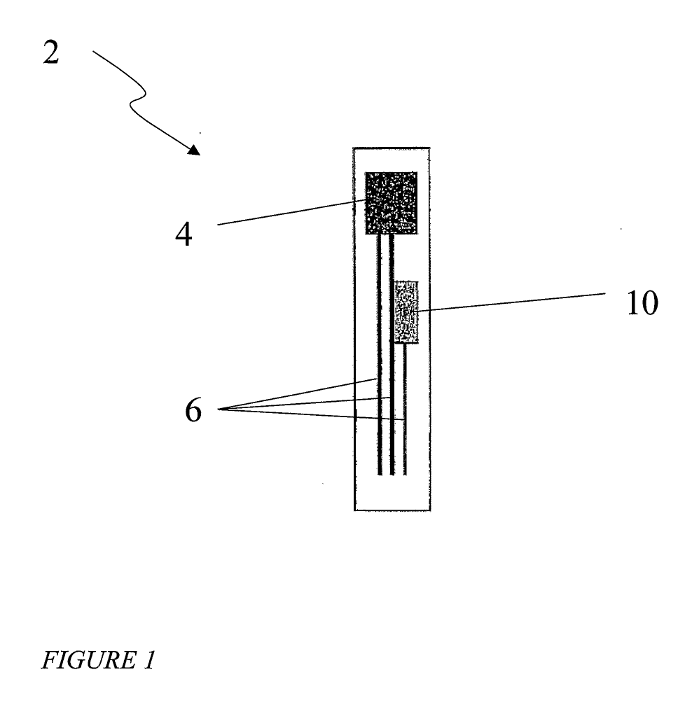

FIG. 1 shows a test element strip or test strip 2 having a sample area 4, electrical tracks 6, and a Radio Frequency Identification (RFID) tag 10. RFID (Radio Frequency Identification) is a technique which is able to carry data in suitable transponders, generally known as tags, and to retrieve data, by machine-readable means, at a suitable time and place to satisfy particular application needs.

An example RFID system may have, in addition to at least one tag, a transceiver or means of reading or interrogating the tags and optionally means of communicating the data received from a tag to an information management system. Transceivers are also known as interrogators, readers, or polling devices. Typically the system may also have a facility for entering or programming data into the tags. RFID tags contain an antenna and an integrated circuit. Various configurations of RFID tags are currently available in the marketplace and one such supplier is Texas Instruments® and the RI-I11-112A tag.

Communication of data between tags and a transceiver is by wireless communication. Such wireless communication is via antenna structures forming an integral feature in both tags and transceivers. During operation, the transceivers transmit a low-power radio signal, through its antenna, which the tag receives via its own antenna to power an integrated circuit. Using the energy it gets from the signal when it enters the radio field, the tag briefly converses with the transceiver for verification and the exchange of data. Once the data is received by the reader, it is sent to a controlling processor in a computer for example, for processing and management.

RFID systems have pre-defined distance ranges over which tags can be read, which depend on several factors such as size of the antenna in the tag, size of the antenna in the transceiver, and the output power of the transceiver. Typically, passive RFID tags operate in the 100 KHz to 2.5 GHz frequency range. Passive RFID tags are powered from the transceiver, whereas active RFID tags have a power source such as a battery, which powers the integrated circuit.

Data within a tag may provide identification data for an item in manufacture, goods in transit, a location, the identity of a vehicle, an animal or user. By including additional data the tags can support applications through item specific information or instructions immediately available on reading the tag. For example, the colour of paint for a car body entering a paint spray area on the production line, or the diabetes testing requirements of an user e.g. on polling of the tag on the first test strip of the day, a user can be informed by the meter that he requires a further three glucose measurements during the next 24 hours.

Transmitting data is subject to the influences of the media or channels through which the data has to pass such as the air interface. Noise, interference and distortion are sources of data corruption that arise in the communication channels that must be guarded against in seeking to achieve error free data recovery. To transfer data efficiently via the air interface that separates the two communicating components requires the data to be modulated with a carrier wave. Typical techniques for modulation are amplitude shift keying (ASK), frequency shift keying (FSK) or phase shift keying (PSK) techniques.

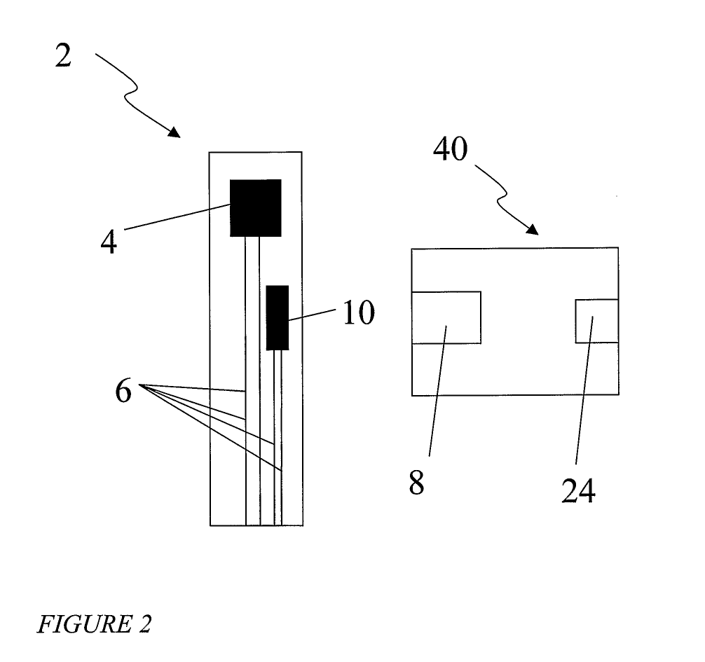

FIG. 1 shows a schematic plan view of test strip 2 of an auto calibration system as will be described hereinafter. Typically test strip 2 may be sized or shaped to fit into a slot on a meter 40 (see FIG. 2). The strip includes an area 4 within which a patient's blood or ISF interacts with bio-reactive elements e.g. enzymes. This reaction causes a change in current on the conductive tracks 6 which is measured. The conductive tracks 6 may be configured to switch the meter on during insertion as will be described hereinafter. The meter 40 contains a means such as a transceiver including an RF source for polling or communicating with RFID tags. RFID tag 10 is fixed to the test strip 2 by means of pressure sensitive or heat seal or cold cure adhesive or alternatively printed on test strip 2 using e.g. carbon tracks during the manufacturing stage of the strip 2. For example, a coil in the RFID tag may be printed by screen printing a conductive track e.g. carbon, gold, silver in the form of a coil. The RFID tags can be written with calibration data, batch number, and expiry data or other data using RF encoding technology after the strip has been manufactured.

The RFID tag can be placed in line on the tracks 6 so that during the activation and measurement of the fluid or during initial insertion the current also activates the RFID tag to cause it to transmit. Alternatively or in addition the RFID tag can be polled by exciting the tag via the transceiver both when the strip is in the meter and when the strip is not in the meter.

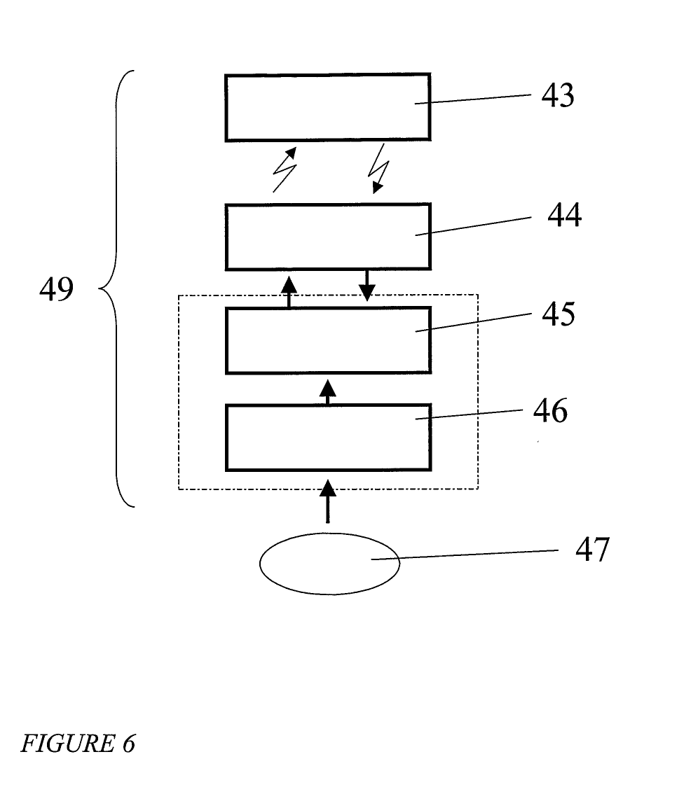

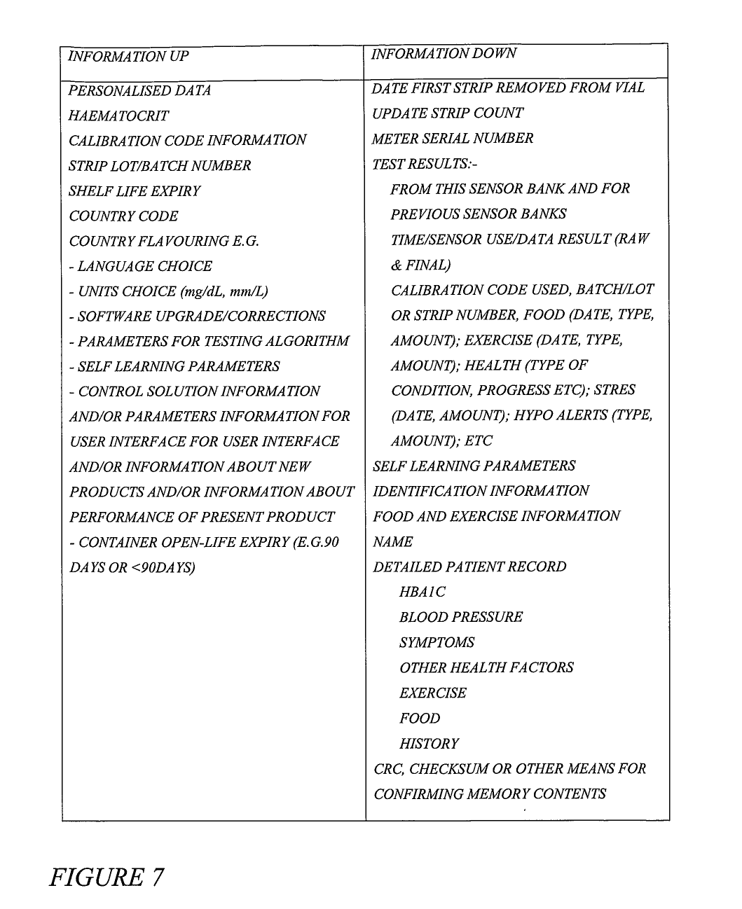

Referring to FIG. 1, the single use test strip 2 has an RFID tag 10 containing information pertaining to batch number, and/or specific calibration data, and, optionally, other information such as ‘expiry date of strips’ information. Examples of information which can be obtained in an RFID tag are shown in the table in FIG. 7. Optionally, before inserting the strip 2 into the meter, the user of the meter activates the meter to a pre-fully functional mode for example by pushing a button. When in this mode, the meter polls for the RFID tag 10 on the nearest test strip. Alternatively, the strip 2 is inserted and the meter switched on (by strip insertion to close a contact or otherwise). The strip 2 may also activate the meter on insertion into the strip port connector 8, 18 by using a conductive track 6 on the strip 2 which forms a bridge between two conductors inside the meter itself. Once the meter is switched on it polls wirelessly for the RFID tag 10 closest to its transceiver. Thus, the RFID tag 10 on the test strip transmits the encoded information such as calibration information and/or batch number and/or expiry date and/or other information as described herein to the meter. Alternatively the tag 10 can be read via RF whilst the strip is in meter, before, during or after blood is deposited on the sample area.

In an example system there is a meter and disposable test strip 2. The system containing a proximity interrogation system including a transceiver, a transponder (an RFID tag), and data processing circuitry. The transceiver includes a microprocessor, a transmitter, a receiver, and a shared transmit/receive antenna. The tag 10 is typically passive (having no on-board power source, such as a battery) and includes an antenna typically configured as a coil, and a programmable memory. As the tag 10 receives its operational energy from the reader, the two devices must be in close proximity. In operation, the transceiver generates sufficient power to excite the tag.

The polling for the RFID tag can either be continuous or activated by the user to enter a pre-fully functional status. When RF energy emanating from the reader's antenna impinges on the tag while it is in close proximity to the tag, a current is induced in the coil of the antenna. The tag does not need to be in line-of-sight of the meter and can typically operate in the range of a few centimetres or up to a few meters in circumstances as will be understood by persons skilled in the art. Alternatively, a transceiver having an antenna in a form of an array could be utilised which would increase the effectiveness of polling of the tag by increasing the angular range of communication. The induced current in the coil of the antenna is routed to the programmable memory of the tag, which then performs an initialization sequence. The transceiver transmits its energy transmitting interrogation signal to the tag and the memory in the tag begins to broadcast its identity and any other requested information over the tag antenna. Information transmitted to the transceiver is decoded as described below.

The transceiver in the meter, picks up the signal from the RFID 10 tag and the transmitted data is used in the processing of the test strip. Circuitry in the meter decodes and processes information received from the RFID tag 10. The strip 2 is inserted into a port 8 on a meter. A user lances a suitable site for example a finger or forearm or palm, and deposits blood or ISF on the sample area 4 on the strip 2. A measurement is made by the following method for example. A voltage is applied to test sensors within sample area 4 on the strip 2 and a current measurement is made. Calibration data is received from the tag 10 specific to strip 2 and is used for calculating the blood glucose level. This level is communicated to the user on the meter display.

The meter may record when the first strip of that container is used. This can be used to calculate information for informing the user how long the vial has been opened, and if a use is recorded each time a strip is used, how many strips remain in a vial or cartridge. Thus, the circuitry in the meter can record the number of strips in a vial from strip information from the tag and then subtracts one from this number every time a strip is used from a specific batch of strips. This information combined with the batch number can be useful for a diabetic to either request additional strips from his physician or to calculate how fast a vial of strips is used over a period of time.

In case the RFID tag becomes damaged during the manufacturing process or during the transit to, e.g. the user, and cannot be read by the meter, or the battery level of the meter is too weak to poll for the RFID tag, the meter has circuitry for allowing a direct manual input of the calibration code. Indeed such direct manual entry can be provided as an option in any event. Typically, the calibration code would be printed on the side of the vial and the user could enter the calibration code before testing commenced. This would allow the user to continue using the strips, thus avoiding having potentially to discard a batch of strips because of a lack of calibration information due to a problem with the RFID tag.

FIG. 2 shows a test strip 2 having a sample area 4, conductive tracks 6, an RFID tag 10, and a meter having a strip port connector 8, and a wireless transceiver 24.

Alternatively as seen in FIG. 2, the RFID tag 10 can be fixed to the test strips and to tracks 6 during manufacture. FIG. 2 shows a test strip 2 having a sample area 4, conductive tracks from the sample area 6 to an edge of test strip 2, and an RFID tag 10. A schematic of a typical meter is also shown which has a strip port connector 8 which is dimensioned to receive a strip 2. The meter also contains a wireless transceiver 24 which polls for information from the RFID tag 10. Conductive tracks emanate from the RFID tag to the edge of the test strip 2. Conductive tracks 6 to RFID tag provide the facility to write calibration code data, expiry of strip data, batch number to the strip 2 during manufacture i.e. to allow the manufacturer to determine the calibration code data of strip 2 after manufacture and write directly to the tag after manufacture of the strip 2.

The application of a hard wired RFID tag 10 as shown in FIG. 2 allows the calibration code data for each batch to be determined after the manufacturing process has been completed i.e. after the constituent parts of the basic strip are in place. The calibration code is then written into the memory of the RFID tag 10 using the electrical tracks 6 on the strip. Alternatively, or in addition in the same way as in FIG. 1, the RFID tags can be written with calibration data, batch number, and expiry data using RF encoding technologies after the strip has been manufactured. Alternatively, or in addition, the tag can be written to (with calibration data) and fixed or stuck onto strip 2 after the basic strip has been made.

During glucose testing, the diabetic inputs the test strip 2 into the meter. The diabetic lances himself and blood from his e.g. finger is drawn to the sample area of the strip. The meter is activated on insertion of the test strip 2 and current is applied to the reactive region of the strip. The meter either polls the RFID tag 10 for the calibration data, batch number, expiry date or alternatively the meter obtains calibration data, batch number, expiry date by using the tracks on the strip. This is a useful design feature of strips since if the meter has reduced power supply i.e. nearly life expired batteries or when a meter is being used in an RF noisy environment which may interfere with the polled RF signal transmission from and to the RFID tag, then the meter can still operate and obtain the calibration code for each batch of strips. Strips with an RFID tag hard wired or coupled through RF technologies, allows the user the option to check the validity of the calibration codes presented on the meter display or to cross check with calibration data presented on manufacturers' vials. Indeed, by producing both a hardwire connection to the RFID tag 10 and an RF connection to the RFID tag 10 from the meter, there is less scope for error in supplying the calibration code to the meter should one connection fail, or as a cross check.

The exemplary embodiments of the invention can be used with integrated lancing/test strip devices such as those described in U.S. Pat. No. 6,706,159. When the meter is activated with the strip 2 inserted into the meter, the meter polls the RFID tag 10 for information specific to that strip 2 such as calibration code data and/or any other information as shown in FIG. 7. The data is then passed to the meter processor. A voltage is applied to the strip 2 and the current versus time data is read by the meter which calculates the glucose value. This glucose value is calculated using the calibration data and an algorithm or a combination thereof and then presented in the form of visual, auditory display.

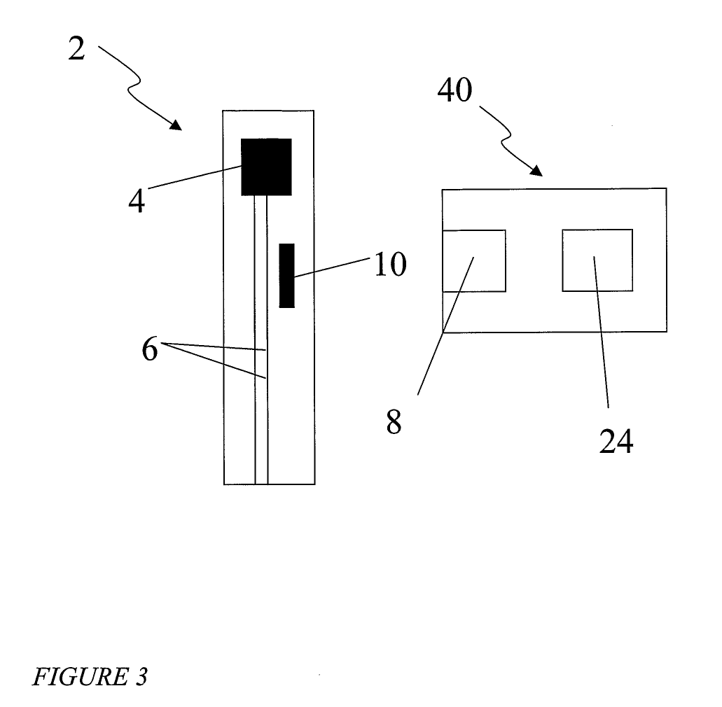

FIG. 3 shows a test strip 2 having a sample area 4, conductive tracks 6 from the sample area 4 to a short edge of test strip 2, and an RFID tag 10. A schematic of a typical meter is also shown which has a strip port connector 8 dimensioned to receive a strip 2. The meter also contains a wireless transceiver 24 which polls for information from the RFID tag 10, when the meter is activated. Meter activation is either by insertion of a test strip 2 as hereinbefore described or by manual depression of a button. Information can be written to the RFID tag via RF only either prior to or after fixing of the tag to test strip 2.

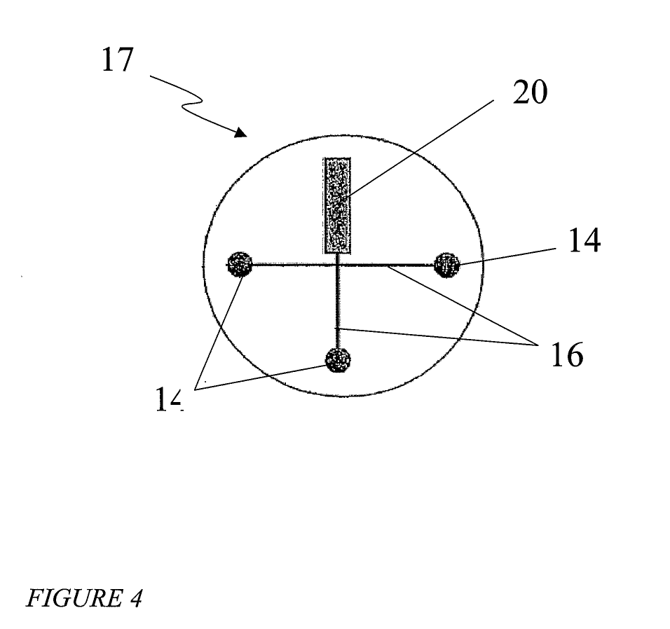

FIG. 4 shows a multi use test strip or module 12 in the form of a disc having three sample areas 14, conductive tracks 16, and an RFID tag 20. An RFID tag 20 is fixed to the test strip. The RFID tag can be activated to release information pertaining to calibration data and/or batch number and/or expiry of test strips 2 or other information as shown in FIG. 7 by providing a transceiver for example in a local controller or separate meter which transmits an appropriate RF field to activate the tag.

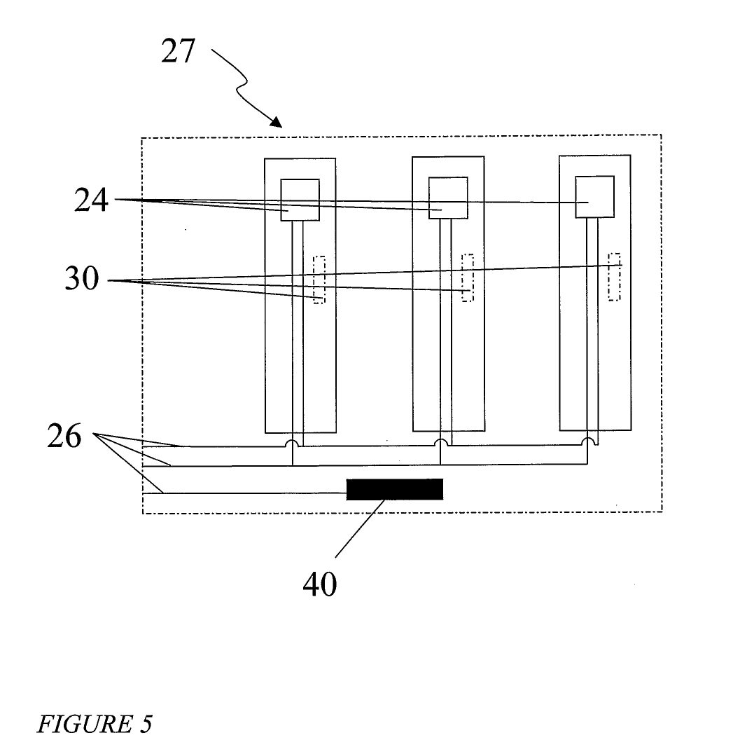

FIG. 5 shows a series of test strips 27 formed as an array for example on a card or in a housing. An RFID tag 40 is attached to the test strip housing which contains information pertaining to calibration data and/or batch number and/or expiry of test strips and/or any other information as shown in FIG. 12. Alternatively or in addition, the strips within the housing may contain two or more RFID tags for example individual RFID tags 30, one associated with each strip 2. Providing two or more tags introduces redundancy. This means that if one of the RFID tags becomes damaged, an alternative RFID tag can be used. Thus, there would be no need to discard that array of strips.

FIG. 6 shows a system 49 in accordance with the present invention for extracting a bodily fluid sample (e.g., an ISF sample) and monitoring an analyte (for example, glucose) and includes a sampling device or cartridge (encompassed within the dashed box), a local controller module 44, and a remote controller module 43, a region of skin for sampling 47, a sampling module 46, and an analysis module 45.

A patient who controls his diabetes through continuous monitoring techniques would normally have a needle or similar attached to his skin. Blood or ISF is periodically or continuously pumped through the needle device to the continuous or multi use test strip 12 attached to the skin. In one embodiment, the continuous or multi use test strip 12 allows the diabetic to monitor his glucose levels without the daily repetitive lancing of his skin, which as previously discussed is a potentially limiting factor in testing due to several issues.

Before use of the continuous or multi use test strip module 12 the patient or user applies the module to his skin. The module is fixed in place either using adhesive or adhesive strip or a strap. A small power source such as button cell is affixed to the sampling module 46. This button cell generates the voltage required for the reaction to take place and to provide an electrical signal to the meter. The current developed at the sensor region 14, 24 in multi-use module 17, 27 is measured by the local controller 44. Once the local controller 44 has measured has measured the current, or the current versus time data, the local controller 44 polls a tag on the test module to obtain, typically at least calibration code information. Using the measured data and the calibration code data the local controller 44 calculates the glucose level. The local controller 44 would typically be attached to the diabetic on his belt. The current or current versus time data is sent to the meter via RF when requested to do so by an RF interrogation signal from the meter. For example the power source can also power a small transmitter in the local controller module 44 as well as the test strip 17, 27. Alternatively, power for either or both of these activities can be extracted from the RF interrogation signal.

The user is informed of the glucose reading optionally initially through a vibration alert device and then through traditional notification means such as LCD display, sound alerts, voice alerts, or Braille instruction or a combination of these or simply through an audio alert and then a visual display.

Alternatively, the result of the measurement can be written into the RFID tag rather than being sent directly to the meter processor via wire or RF. This is now described in more detail. The multi use test strip is applied to the skin for continuous measurement techniques and has at least one writeable RFID tag 20, 30, 40 as shown in FIGS. 4 and 5. Data is written into the memory of the RFID tag using a small battery contained either within the multi use module itself or within a separate attached local controller (see item 44 in FIG. 4). For example, during sampling of the analyte, the test module 17, 27 is supplied with a voltage by the power source which generates a measurement signal (e.g. current, current versus time etc). The measurement signal is then written to the RFID tag 20, 30, 40 ready to be read by a local controller module 44 (or remote controller module 43) using a transceiver. Remote controller module 43 can instead read data from local controller 44. The RFID tag 20, 30, 40 contains strip specific calibration data, expiry date, and batch information and/or any other information from FIG. 5, as well as the newly written measurement data. When the diabetic requires a glucose reading using this embodiment, the meter circuit (e.g. within remote controller module 43) would poll the RFID tag 20, 30, 40 and enter into a test reading mode. Calibration data and/or any other information from FIG. 7 would then be downloaded into the remote controller module, and the measurement information would also be downloaded into the meter. This would allow the user to check his blood glucose reading at any point in the day.

During use of a multi use strip 17 or an array of strips 27, one of the RFID tags 20, 30, 40 contained within the array or combinations thereof transmit the calibration data, expiry date of strips, batch number or other information when a transceiver polls for a tag and excites the tag to transmit such data. A monitoring device for example a remote controller (see 43 in FIG. 6) could be used as a parental monitor. The remote control or parental monitor comprises a transceiver, a modem for communication to the Public Switched Telephone Network (PSTN), a processor and circuitry to control and act upon predefined alarm conditions. When a sampling area 14, 24 on the multi use test strip or array of strips 17, 27 has been filled with a sample such as blood and a voltage applied to sampling electrodes (such as a working and counter/reference electrode) within sampling area 14, 24), an electrochemical reaction in the strip takes place. Either the current developed in the sample after a predetermined amount of time or the current versus time profile generated can be written into the memory of the RFID tag. Other types of measurement and reactions such as calorimetric or photometric and so on can be applicable to the present invention as would be understood by someone skilled in the art. During polling of the tag by a remote monitor e.g. a parental monitor, data held within the tag including for example calibration code data and measurement data and optionally other data as referred to in FIG. 7 is transmitted back to the parental monitor. The glucose value is then calculated within the remote controlled using this data. The remote control (e.g. parental monitor contains an alarm (not shown) which can be configured to operate in a number of ways.

Of course, the remote control or parental module may establish a communications link with, instead of the public switched telephone network, any other circuit-switched communications network, or a mobile telephony network, the internet or another packet-switched communications network. Alternatively, the local controller may establish a communications link with a mobile telephony network or another wireless communications network for exactly the same purpose.

Pre-defined alarm thresholds within the parental monitor can be triggered. These alarm thresholds can include glucose levels at a certain time of day, or glucose levels compared to dietary intake. Similarly, alarm levels thresholds can show that an RF link has been lost (or never made) or that data has not been updated correctly and/or in a timely fashion. Furthermore, the alarm functionality can optionally be controlled by the patient. For example, the alarm could have a range of opt in or opt out functions. An example opt in function is the ‘sleep monitor mode’. In this mode during sleep if the glucose level falls or rises above a set threshold, then the alarm could be activated to inform the patient, alternatively or in addition an automatic dial out facility could be provided. Thus, if a diabetic could not be roused to respond to the alarm, the remote control module could ‘dial out’ to inform a third party of the alarm condition. For example, the modem can be used either to dial out to a ‘best friend’ or with a recorded message stating the alarm condition, for example, that the diabetic of a named address is in a life threatening condition requiring urgent medical attention, or alternatively, to dial out to another designated number such as to a response centre or the emergency services again with the same or similar message. Indeed, the dial out number to the response centre such as the emergency services could be designated as the first dial out number to be dialled with the ‘best friend’ being informed thereafter.

A wireless alternative can be integrated within remote control monitor or the parental monitor. Such wireless alternative could be in the form of a mobile phone and as such, the connection to the response centre using either connection means would be seamless. This introduction of redundancy to the parental monitor is a useful feature and can give further reassurance to a patient that assistance would automatically be obtained in the event that the patient enters a hypoglycaemic or hyperglycaemic state.

The parental monitor can be either wearable or wall mounted or both, for example can be designed both for use as a removable wearable unit and for interaction with a docking station. It would be especially useful if the parental controller was wall mountable, since diabetics might want to continuously test their glucose levels whilst in bed, or a physician might want to use such a system when he wishes to monitor a diabetic over a period of time, for example in hospital or during a period of low physical inactivity such as sleeping. The activation of the modem in the instance of a patient being under observation be it at hospital or at home or if say the patient could not meet his specialist physician face to face by being in a regional hospital which would not normally employ specialist physicians would be especially useful. In this respect, the parental monitor could then send glucose readings and/or dietary intake and/or other data to the remote physician using the modem in combination with the PSTN.

FIG. 7 details in addition to the information listed above the types of information which might be uploaded from an RFID tag to the meter and the types of information which might be written back down from the meter to the RFID tag for later use by a patient or clinician, or for use during further testing in any of the embodiments.

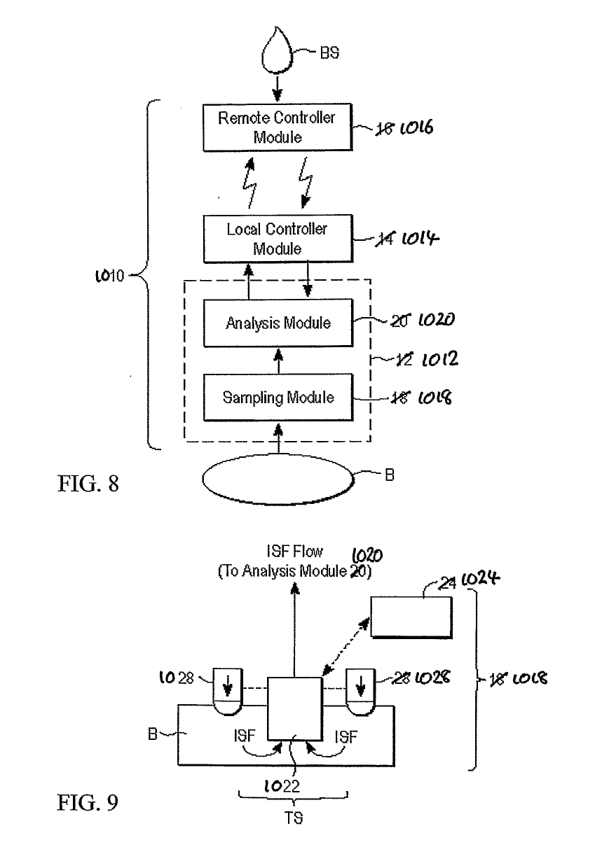

The above being a general description of glucose metering systems according to the invention, there now follows a more detailed description of two example systems. The first system is illustrated in FIGS. 8-10 and described in more detail in U.S. patent application Ser. No. 10/882,994. The system 1010 includes a disposable cartridge 1012 (encompassed within the dashed box), a local controller module 1014, and a remote controller module 1016, as illustrated in FIG. 8.

In system 1010, disposable cartridge 1012 includes a sampling module 1018 for extracting the bodily fluid sample (namely, an ISF sample) from a body B, e.g., a user's skin layer, and an analysis module 1020 for measuring an analyte (i.e., glucose) in the bodily fluid. Sampling module 1018 and analysis module 1020 may be any suitable sampling and analysis modules known to those of skill in the art. Examples of suitable sampling and analysis modules are described in International Patent Application WO 02/49507. However, in system 1010, sampling module 1018 and analysis module 1020 are both configured to be disposable since they are components of disposable cartridge 1012.

As depicted in FIG. 9, the particular sampling module 1018 of system 1010 is, however, an ISF sampling module that includes a penetration member 1022 for penetrating a target site (TS) of body B and extracting an ISF sample, a launching mechanism 1024 and at least one pressure ring 1028. ISF sampling module 1018 is adapted to provide a continuous or semi-continuous flow of ISF to analysis module 1020 for the monitoring (e.g., concentration measurement) of an analyte (such as glucose) in the ISF sample.

During use of system 1010, penetration member 1022 is inserted into the target site (i.e., penetrates the target site) by operation of launching mechanism 1024. For the extraction of an ISF sample from a user's skin layer, penetration member 1022 may be inserted to a maximum insertion depth in the range of, for example, 1.5 mm to 3 mm. In addition, penetration member 1022 may be conFigured to optimize extraction of an ISF sample in a continuous or semi-continuous manner. In this regard, penetration member 1022 may include, for example, a 25 gauge, thin-wall stainless steel needle (not shown in FIG. 8 or 9) with a bent tip, wherein a fulcrum for the tip bend is disposed between the needle's tip and the needle's heel. Suitable needles are described in U.S. Pat. No. 6,702,791 and US Patent Application no. 2003/0060784 (Ser. No. 10/185,605) which are hereby fully incorporated by reference.

Launching mechanism 1024 may optionally include a hub (not shown in FIG. 8 or 9) surrounding penetration member 1022. Such a hub is configured to control the insertion depth of penetration member 1022 into the target site. Insertion depth control may be beneficial during the extraction of an ISF sample by preventing inadvertent lancing of blood capillaries, which are located relatively deep in a user's skin layer, and thereby eliminating a resultant fouling of an extracted ISF sample, clogging of the penetration member or clogging of an analysis module by blood. Controlling insertion depth may also serve to minimize pain and/or discomfort experienced by a user during use of system 10.

Although FIG. 9 depicts launching mechanism 1024 as being included in sampling module 1018, launching mechanism 1024 may optionally be included in disposable cartridge 1012 or in local controller module 1014 of system 1010. Furthermore, to simplify employment of system 1010 by a user, sampling module 1018 may be formed as an integral part of the analysis module 1020.

In order to facilitate the extraction of a bodily fluid (e.g., ISF) from the target site, penetration member 1022 may be arranged concentrically within at least one pressure ring 1028. Pressure ring(s) 1028 may be of any suitable shape, including but not limited to, annular. An example of such an arrangement is disclosed in U.S. Pat. No. 5,879,367 which is hereby fully incorporated by reference.

During use of system 1010, pressure ring 1028 is applied in the vicinity of the target site TS, prior to penetration of the target site by penetration member 1022, in order to tension the user's skin layer. Such tension serves to stabilize the user's skin layer and to prevent tenting thereof during penetration by the penetrating member. Alternatively, stabilization of the user's skin layer prior to penetration by the penetrating member may be achieved by a penetration depth control element (not shown) included in sampling module 1018. Such a penetration depth control element rests or “floats” on the surface of the user's skin layer, and acts as a limiter for controlling penetration depth (also referred to as insertion depth). Examples of penetration depth control elements and their use are described in U.S. patent application Ser. No. 10/690,083 and EP 1,527,736, which are hereby fully incorporated herein by reference.

Once penetration member 1022 has been launched and has penetrated the target site TS, a needle (not shown in FIG. 8 or 9) of penetration member 1022 will reside, for example, at an insertion depth in the range of about 1.5 mm to 3 mm below the surface of the user's skin layer. If desired, penetration member 1022 may be launched coincidentally with application of pressure ring(s) 1028 to the user's skin layer, thereby enabling a simplification of the launching mechanism. The pressure ring(s) 1028 applies/apply a force on the user's skin layer (indicated by the downward pointing arrows of FIG. 9) that pressurizes ISF in the vicinity of the target site. A sub-dermal pressure gradient induced by the pressure ring(s) 1028 results/result in flow of ISF up the needle and through the sampling module to the analysis module (as indicated by the curved and upward pointing arrows of FIG. 9).

ISF flow through a penetration member's needle is subject to potential decay over time due to depletion of ISF near the target site and due to relaxation of the user's skin layer under the pressure ring(s) 1028. The systems and methods of the present invention address this by varying one or more aspects of the applied pressure.

In one variation, the amount of applied pressure may be varied over a given time. While contact between the pressure ring(s) and the skin might be constant, the amount of that pressure may be varied. For example, the amount of pressure may be progressively increased proportionately or otherwise to the volume or flow rate of the ISF being extracted. Alternatively, the increase in pressure may be staggered or applied in a step-wise fashion. Still yet, the pressure may be oscillated between various levels of greater and lesser pressure, where the reduction in pressure may include the discontinuance of pressure by completely removing the pressure ring(s) from contact with the skin. The oscillation frequency of the pressure ring(s) may be constant or varied depending on the application. For example, the application times of higher pressure (“on”) and lower or no pressure (“off”) may be the same (e.g., 3 minutes on followed by 3 minutes off, etc.) or different (e.g., 15 minutes on followed by 10 minutes off, etc.) or one may be constant and the other may vary (e.g., 15 minutes on followed by 20 minutes off followed by 15 minutes on followed by 10 minutes off, etc.).

In other variations, while the amount of applied pressure to the skin may be constant over a period of time, the location of that pressure relative to the needle penetration site may vary over time. For example, the initial pressure may commence at a certain radial distance (assuming a substantially annular configuration of the pressure ring) from the penetration site where that radial distance is reduced or increased over time. The change in distance may be gradual or less so depending on the application or in response to ISF extraction flow or volume. This may be accomplished by the use of multiple pressure rings having varying diameters which are individually and successively applied to the target site.

The location of the initial pressure may be maintained, but the radial surface area over which the pressure is applied may be increased or decreased. In other words, the amount of surface area of the pressure ring in contact with the skin may be increased or increased. This may also be accomplished by the use of multiple pressure rings which are individually but cumulatively applied or successively removed from application to the skin.

Returning to FIGS. 8-11, and as mentioned above, pressure ring(s) 1028 may be applied to the user's skin layer in an oscillating manner (e.g., with a predetermined pressure ring(s) cycling routine or with a pressure ring cycling routine that is controlled via and is responsive to ISF flow rate measurement and feedback) while the penetration member is residing in the user's skin layer in order to minimize ISF flow decay. In addition, during application of pressure in an oscillating manner, there may be time periods during which the pressure applied by the pressure ring(s) is varied or the local pressure gradient is removed and the net outflow of ISF from the user's skin layer is eliminated. In addition, pressure ring(s) 28 may be configured to apply an oscillating mechanical force (i.e., pressure) in the vicinity of the target site while the penetration member is residing in the user's skin layer. Such oscillation may be achieved through the use of a biasing element (not shown in FIG. 8 or 9), such as a spring or a retention block.

Any suitable glucose sensor known to those of skill in the art may be employed in analysis modules according to the present invention. Glucose sensor 1310 may contain, for example, a redox reagent system including an enzyme and a redox active compound(s) or mediator(s). A variety of different mediators are known in the art, such as ferricyanide, phenazine ethosulphate, phenazine methosulfate, pheylenediamine, 1-methoxy-phenazine methosulfate, 2,6-dimethyl-1,4-benzoquinone, 2,5-dichloro-1,4-benzoquinone, ferrocene derivatives, osmium bipyridyl complexes, and ruthenium complexes. Suitable enzymes for the assay of glucose in whole blood include, but are not limited to, glucose oxidase and dehydrogenase (both NAD and PQQ based). Other substances that may be present in the redox reagent system include buffering agents (e.g., citraconate, citrate, malic, maleic, and phosphate buffers); divalent cations (e.g., calcium chloride, and magnesium chloride); surfactants (e.g., Triton, Macol, Tetronic, Silwet, Zonyl, and Pluronic); and stabilizing agents (e.g., albumin, sucrose, trehalose, mannitol and lactose).

In an embodiment in which the analysis module includes an electro-chemical based glucose sensor, the specific structure of the electrochemical sensor will depend on the nature of the analyte. In general, however, each device will include an electrode layer and at least one reagent layer deposited on a substrate. As used in the specification and claims hereof, the term “layer” refers to a coating applied to all or part of the surface of the substrate. A layer is considered to be “applied to” or “printed on” the surface of the substrate when it is applied directly to the substrate or the surface of a layer or layers previously applied to the substrate. Thus, deposition of two layers on the substrate may result in a three layer sandwich (substrate, layer 1, and layer 2) or in the deposition of two parallel tracks, as well as intermediate configurations with partial overlap.

The substrate used may be any dimensionally stable material. In general the substrate will be an electrical insulator, although this is not necessary if a layer of insulation is deposited between the substrate and the electrodes. The substrate should also be chemically compatible with the materials which will be used in the printing of any given sensor. This means that the substrate should not significantly react with or be degraded by these materials, although a reasonably stable print image does need to be formed. Specific examples of suitable materials include polycarbonate and polyester.

The electrodes may be formed of any conductive material which can be deposited in patterns. This would include carbon electrodes and electrodes formed from platinized carbon, gold, silver, and mixtures of silver and silver chloride. Insulation layers are deposited as appropriate to define the sample analysis volume and to avoid a short circuiting of the sensor. Insulating materials which can be printed are suitable, including for example polyester-based inks.

It will be well understood that this structure causes the generation of both charge and current in the presence of an analyte, allowing for the following to be measured: an inter-electrode impedance; an inter-electrode current; a potential difference; an amount of charge; a change over time of any of the aforesaid; any combination of the aforesaid; or any other indicator of the amount of electricity passing from one electrode to another, or the extent to which exposure of the sensor to the fluid generates electrical energy or electrical charge or otherwise affects the electrical characteristics of the sensor. Local controller module 14 may measure any of these things, preferably electrical current, via electrical contacts (not shown) and convert it to one that is representative of the ISF glucose concentration.

Other embodiments use photometric or calorimetric sensors comprising a substrate and at least a first reagent including a catalyst and a dye or dye precursor and the catalyst catalyses, in the presence of the analyte, the denaturing of the dye or the conversion of the dye precursor into a dye. For glucose sensors, the preferred combination is a combination of glucose oxidase and horseradish peroxidase as a catalyst and leuco-dye as a dye precursor. The leuco-dye may, for example, be 2,2-azino-di-[3-ethylbenzthiazoline-sulfonate], tetramethylbenzidine-hydrochloride or 3-methyl-2-benzothiazoline-hydrazone in conjunction with 3-dimethylamino-benzoicacide. The reagent may be laid down as a film or membrane over a opening in a substrate or over a portion of a substrate or placed into a chamber in a substrate as in WO02/49507.

It is well understood that this combination of enzyme and leuco-dye causes the colour or depth of colour of the reagent layer to change in the presence of glucose, allowing for the following to be measured: opacity; transparency; transmissivity reflectivity or absorptivity; a transmission, reflection or absorption spectrum, peak, gradient or ratio; any one of more parts of such a spectrum; colour; a change over time of any of the aforesaid; and any combination of the aforesaid.

If a fluorophore is used instead of a non-fluorescing leuco-dye, the amount of glucose can be determined by looking at the fluorescence properties of the reagent, such as: fluorescence intensity; emissivity; an emission or excitation spectrum, peak, gradient or ratio; any one of more parts of such a spectrum; an emission polarization; an excited state lifetime; a quenching of fluorescence; a change over time of any of the aforesaid; or any combination of the aforesaid.

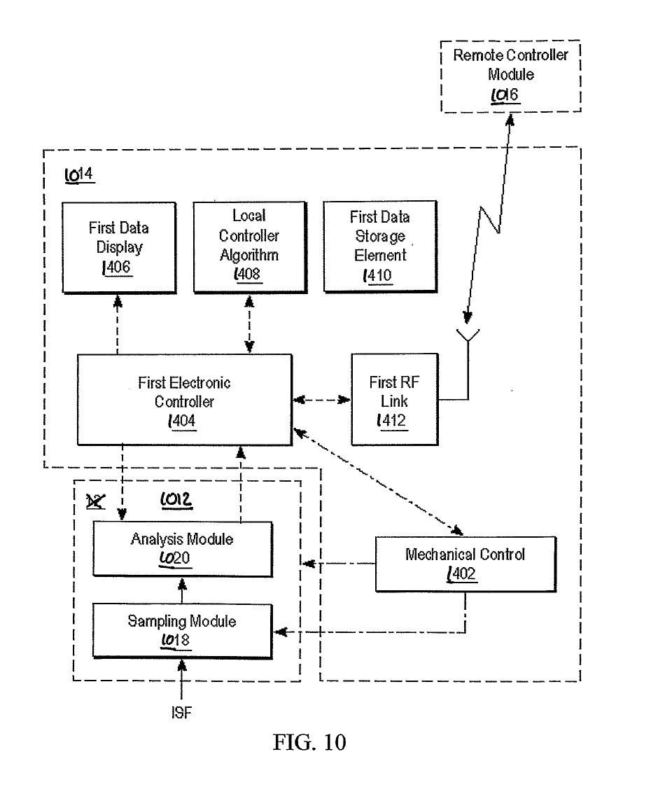

Local controller module 1014 is depicted in simplified block form in FIG. 10. Local controller module 1014 includes a mechanical controller 1402, a first electronic controller 1404, a first data display 1406, a local controller algorithm 1408, a first data storage element 1410 and a first RF link 1412. Local controller module 1014 is configured such that it may be electrically and mechanically coupled to disposable cartridge 1012. The mechanical coupling provides for disposable cartridge 1012 to be removably attached to (e.g., inserted into) local controller module 1014. Local controller module 1014 and disposable cartridge 1012 are configured such that they may be attached to the skin of a user by, for example, by a strap, in a manner which secures the combination of the disposable cartridge 1012 and local controller module 1014 onto the user's skin.

During use of system 1010, first electronic controller 1404 controls the measurement cycle of the analysis module 1020, as described above. Communication between local controller module 1014 and disposable cartridge 1012 takes place via the electrical contacts on analysis module 1020 and the corresponding electrical contacts on local controller module 1014. Electrical signals representing the glucose concentration of an ISF sample are then sent by the analysis module to the local controller module. First electronic controller 1404 interprets these signals by using the local controller algorithm 1408 and displays measurement data on a first data display 1406 (which is readable by the user). In addition, measurement data (e.g., ISF glucose concentration data) may be stored in first data storage element 1410. This element may be an RFID tag, in which case FIG. 10 should be understood as though data storage element 1410 and RF link 1412 were merged into a single functional block.

Prior to use, an unused disposable cartridge 1012 is inserted into local controller module 1014. This insertion provides for electrical communication between disposable cartridge 1012 and local controller module 1014. A mechanical controller 1402 in the local controller module 1014 securely holds the disposable cartridge 1012 in place during use of system 1010.

After attachment of a local controller module and disposable cartridge combination to the skin of the user, and upon receiving an activation signal from the user, a measurement cycle is initiated by first electronic controller 1404. Upon such initiation, penetration member 1022 is launched into the user's skin layer to start ISF sampling. The launching may be initiated either by first electronic controller 1404 or by mechanical interaction by the user.

First RF link 1412 of local controller module 1014 is configured to provide bi-directional communication between the local controller module and a remote controller module 1016, as depicted by the jagged arrows of FIGS. 8 and 10. The local controller module incorporates a visual indicator (e.g., a multicolour LED) indicating the current status, e.g., a red light may be used to indicate a hypo or hyperglycaemic state and a green light may be used to indicate a euglycaemic state, etc., of the system.

Local controller module 1014 is configured to receive and store measurement data from, and to interactively communicate with, disposable cartridge 1012. For example, local controller module 1014 may be configured to convert a measurement signal from analysis module 1020 into an ISF or blood glucose concentration value. Alternatively, the conversion might be done by the cartridge 1012. Information stored in local controller module 1014 is preferably stored in a passive RFID tag contained within the module; however, it may be otherwise stored so long as the local controller 1014 has some active or passive RF communications capability by means of which it can communicate with the remote module 1016 when interrogated by the remote module 1016. One form of passive communication is for the local controller 1014 to extract energy from an RF interrogation signal from the remote controller module 1016 to use in the transmission of the information. It may use the same technique to extract energy from an RF testing signal from the remote controller module 1016 to use in the operation of the launching mechanism and/or pressure rings. The testing and interrogation signals may be the same signal, in which case there may be no need to store the data in the RFID tag at all. Another form of passive communication is for the local controller 1014 to modulate or modify the interrogation signal, as with a passive RFID tag.

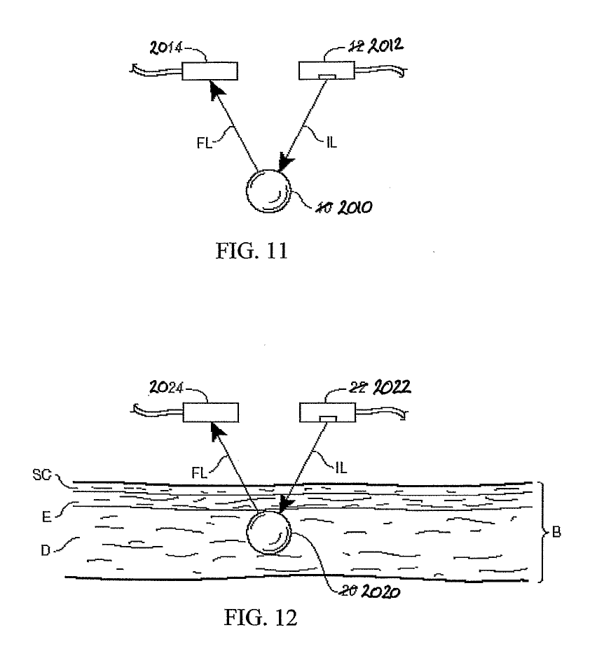

Another system is illustrated in FIGS. 11-13B. FIG. 11 is a simplified schematic illustration depicting interaction between a fluorescent light-emitting bead 2010, light emitter 2012 and light detector 2014 that is relevant to various embodiments of the present invention. Fluorescent light-emitting bead 2010 includes at least one fluorescent reactant (e.g., a fluorescent dye) that emits fluorescent light FL as a result of absorbing incident light IL (that has been emitted by light emitter 2012), with characteristics of the emitted fluorescent light FL being dependent on the concentration of an analyte that is in communication with (e.g., in contact with) the fluorescent light-emitting bead. Fluorescent reactants that can be included in such a fluorescent light-emitting bead, and their behavior when in communication with an analyte, are described in U.S. Pat. Nos. 5,342,789, 6,040,194, and 6,232,130, each of which is hereby fully incorporated by reference. Fluorescent light-emitting bead 2010 can also include an encapsulating material such as, for example, alginate. Preferred fluorescent reagents reversibly bind to the analyte.

When using a fluorescent reagent, the following characteristics can be measured, in this case by transdermal optometry: a fluorescence intensity; an emission or excitation spectrum, peak, gradient or ratio; any one or more parts of such a spectrum; an emission polarization; an excited state lifetime; a quenching of fluorescence; a change over time of any of the aforesaid; any combination of the aforesaid; or any other indicator of the extent to which exposure of the fluorescent reagent to the fluid affects its fluorescence characteristics.

Preferred embodiments use a reagent comprising or labelled with a donor molecule and an acceptor molecule, where the measurable characteristic is an indicator of the extent to which non-radiative fluorescence resonance energy transfer occurs between the donor and the acceptor upon reversible binding of the reagent to the analyte. The reagent may comprise a specific binding pair, one of which is, or is labelled with, the donor molecule and the other of which is, or is labelled with, the acceptor molecule. The sensor may comprise a envelope that contains and is substantially impermeable to the reagent, but is permeable to the analyte. The envelope may also a microdialysis vessel or a microcapsule. Further details of reagents can be found in U.S. Pat. No. 6,040,194, which is hereby fully incorporated by reference.

FIG. 12 is a simplified schematic illustration depicting interaction between a fluorescent light-emitting bead 2020 implanted in a user's body B, a light emitter 2022 and a light detector 2024 that is relevant to various embodiments of the present invention. The portion of user's body B depicted in FIG. 12 includes a Stratum Corneum portion SC, an Epidermis portion E and Dermis portion D.

As with fluorescent light-emitting bead 2010, fluorescent light-emitting bead 2020 includes at least one fluorescent reactant (e.g., a fluorescent dye) that emits fluorescent light FL as a result of absorbing incident light IL (that has been emitted by light emitter 2022), with characteristics of the emitted fluorescent light being dependent on the concentration of an analyte that is in communication with the fluorescent light-emitting bead.

FIG. 12 depicts fluorescent light-emitting bead 2020 implanted in a user's body B. In this circumstance, incident light IL and fluorescent light FL are of a wavelength(s) and intensity such that incident light IL is able to pass through the user's body B to reach fluorescent light-emitting bead 2020 and fluorescent light FL is able to pass through the user's body to reach light detector 2024. Fluorescent light-emitting bead 2020 includes at least one fluorescent reactant and is configured in such a way that a predetermined characteristic(s) of fluorescent light FL varies as a function of bodily fluid analyte concentration (e.g., glucose concentration) in the user' body B.

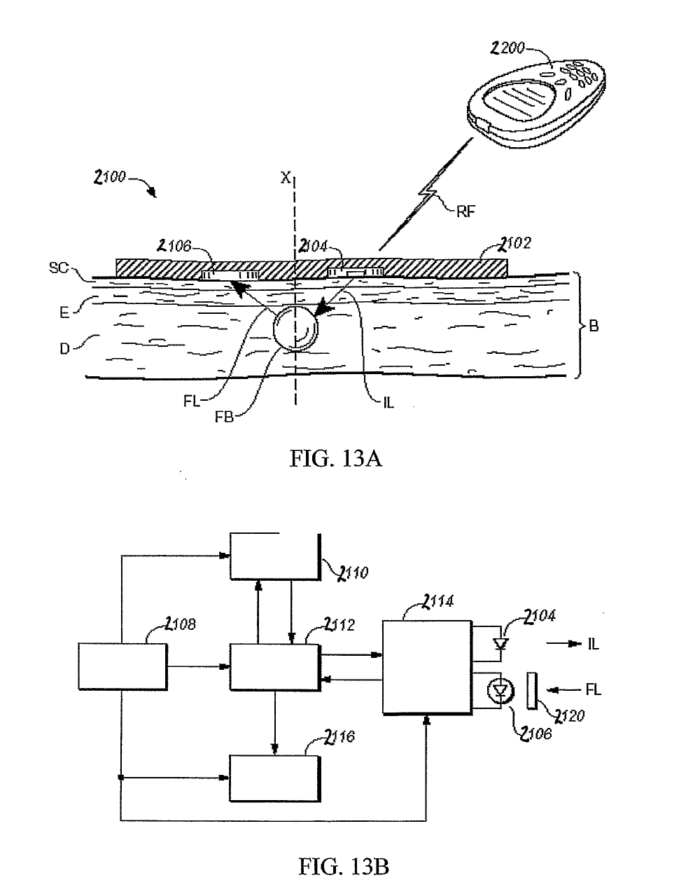

FIG. 13A is a simplified cross-sectional view of an adhesive fluorescence measurement patch 2100 for use with a fluorescent light-emitting bead FB implanted within a user's body B, that includes a Stratum Corneum portion SC, an Epidermis portion E and Dermis portion D, according to an exemplary embodiment of the present invention. In FIG. 13A, adhesive fluorescence measurement patch 2100 is removably adhered to a user's body B and in communication with a remote module 2200 via radio-frequency signals RF. Adhesive fluorescence measurement patch 2100 includes an adhesive sheet 102 configured for removable adhesion to user's body B, a light emitter 2104 attached to adhesive sheet 2102, and a light detector 2106 also attached to adhesive sheet 2102. Although FIG. 13A depicts light emitter 2104 and light detector 2106 embedded in adhesive sheet 102, the attachment of light emitter 2104 and light detector 2106 to adhesive sheet 2102 can take any suitable form known to one skilled in the art.

Fluorescent light-emitting bead FB can be implanted, for example, in the range of approximately 1 mm to 4 mm below the surface of a user's skin. In addition, light emitter 2104 and light detector 2106 can be located, for example, in the range of 0 mm to 10 mm above the surface of the user's skin when adhesive fluorescence measurement patch 2100 is adhered to the user's body B (i.e., adhered to the user's skin).

For the sake of simplicity, FIG. 13A depicts adhesive fluorescence measurement patch 2100 as including only an adhesive sheet, light emitter and light detector. However, once apprised of the present disclosure, one skilled in the art will recognize that adhesive fluorescence measurement patches, kinematic adhesive fluorescence measurement patches and kinematic adhesive fluorescence measurement bands according to the present invention can include various other components, electrical and/or optical, that provide for suitable and beneficial operation. In this regard, FIG. 13B is a simplified schematic diagram depicting the operative interaction of various electrical and optical components, including a light emitter 2104 and a light detector 2106, suitable for use in the adhesive fluorescence measurement patch of FIG. 13A and other embodiments of the present invention. In FIG. 13B, elements or other items common with FIG. 13A are identically labeled.

As depicted in FIG. 13B, the electrical and optical components include a power module 2108, an RF transceiver module 2110, a micro-controller module 2112, a driver/amplifier module 2114, a buzzer module 2116 (for providing feedback to a user) and an optical filter module 2120. Light emitter 2104 can be, for example, an LED 525 nm wavelength light emitter such as SMD LED part number LTST-C903TGKT available from Lite-On Corp. Light detector 2106 can be, for example, light detector part number S 8745-01 available from Hamamatsu. Optical filter module 2120 can include, for example, 600 nm and 700 nm band pass filters. Micro-controller module 2112 can be, for example, an MSP 430 series micro-controller available from Texas Instruments. Power module 2108 can be, for example, a rechargeable or non-rechargeable battery module or a circuit that extracts power from wireless signals from remote controller 2200. If desired, all the electrical and optical components depicted in FIG. 13B can be mounted on a printed circuit board (PCB) and the PCB attached to adhesive sheet 2102.

In addition, once apprised of the present disclosure, one skilled in the art will recognize that embodiments of the present invention can be readily modified for use with suitable fluorescent light-emitting devices other than a fluorescent light-emitting bead. For example, such adhesive fluorescence measurement patches could be used with fluorescent injected oils or fluorescent tattoos as described in U.S. Pat. No. 5,342,789, which is hereby fully incorporated by reference.

In FIG. 13A, fluorescent light-emitting bead FB is implanted in user's body B, and contains at least one fluorescent reactant that emits fluorescent light FL as a result of absorbing incident light IL. In addition, a characteristics) of fluorescent light FL varies as a function of analyte concentration in contact with fluorescent light-emitting bead FB. Therefore, adhesive fluorescence measurement patch 2100, in conjunction with fluorescent light-emitting bead FB and remote module 2200, can be used for measuring the concentration of an analyte (e.g., blood glucose) in the bodily fluid of a user's body.

Referring again to FIG. 13A, an imaginary optical axis X of adhesive fluorescence measurement patch 2100 is depicted by a broken line. Light emitter 2104 and light detector 2106 are attached to adhesive sheet 2102 in a predetermined relationship relative to imaginary optical axis X. In addition, imaginary optical axis X is positioned in a predetermined juxtaposition to the fluorescent light-emitting bead FB when the adhesive fluorescence measurement patch is removably adhered to a user's body (as in FIG. 13A). The predetermined juxtaposition of imaginary optical axis X and fluorescent light-emitting bead FB will typically be associated with a suitable alignment tolerance in the range of, for example, +/−1 mm to +/−2 mm.

The predetermined relationship of light emitter 2104 and light detector 2106 with imaginary optical axis X and the predetermined juxtaposition of imaginary optical axis X with the fluorescent light-emitting bead FB provide for (i) emitted incident light IL from light emitter 2104 to be incident on, and absorbed by, fluorescent light-emitting bead FB and (ii) fluorescent light FL emitted by fluorescent light-emitting bead FB to be detected by light detector 2106 (the emitted light IL and fluorescent light FL are, for the sake of simplicity, depicted as arrows in FIG. 13A (as well as in FIGS. 11 and 12)). Therefore, adhesive fluorescence measurement patch 100 can be readily adhered to user's body B in a position that provides for incident light IL to operatively reach fluorescent light-emitting bead FB, as well as for fluorescent light FL to operatively reach light detector 2106. Since light emitter 2104 and light detector 106 are securely attached to adhesive sheet 2102 in a proper predetermined relationship to imaginary optical axis X, an operable alignment of light emitter 2104 and light detector 2106 with an implanted fluorescent light-emitting bead FB is easily obtained and maintained during use.

It should be noted that although FIG. 13A depicts light emitter 2104 and light detector 2106 as being symmetrically disposed about imaginary optical axis X, such symmetry is not necessarily required. In addition, the predetermined relationship of light emitter 2104 and light detector 2106 with imaginary optical axis X, as well as the predetermined juxtaposition of imaginary optical axis X with the fluorescent light-emitting bead FB, can be such the amount of reflected light from the fluorescent light-emitting bead received by the light detector is relatively minimized while the amount of fluorescent light received by the light detector is relatively maximized.

Adhesive sheet 2102 can be any suitable adhesive sheet known to those of skill in the art including, for example, adhesive sheets that include commercially available pressure sensitive adhesives. Furthermore, adhesive sheets employed in embodiments can include a top layer and at least one adhesive lower layer disposed on at least a portion of the top layer.

The top layer and adhesive lower layer(s) employed in the adhesive sheet can be any suitable combination of single-sided adhesive layers, double-sided adhesive layers, transfer adhesive layers and non-adhesive layers. The single-sided and double-sided adhesive layers can be pressure sensitive, in that they removably adhere to a surface of a user's body when pressure is applied. Typical pressure sensitive adhesive layers include those based on acrylics, natural rubber, synthetic rubber and silicone polymers. Suitable pressure sensitive adhesive layers are commercially available from, for example, Adhesives Research, Inc., of Glen Rock, Pa. under the commercial name ARcare®.

The top layer and adhesive lower layer(s) of an adhesive sheet can be clear or opaque, and are typically flexible. The top layer and adhesive lower layer(s) can be made, for example, from an extruded or cast polymer film, or can be made using woven or non-woven fabric and can be elastic, or inelastic. In addition, they can be made from any suitable material, including, for example polyester, polycarbonate, polystyrene, polypropylene, polyethylene, acrylonitrile butadiene styrene (ABS), polyurethane, silicone, and woven or non-woven fabrics. Suitable polymer films and fabrics can be purchased, for example, from Tekra Corporation of New Berlin, Wis.

If desired, one or more release liners can be employed to cover all or a portion of adhesive sheets employed in embodiments of the present invention. Such release liners are typically made by, for example, siliconizing polyester, polyethylene, polypropylene or paper. Release liners can also be manufactured by treating the surface of a suitable material with a fluorocarbon-based compound. Prior to use of an adhesive fluorescence measurement patch, one or all of the release liners are pealed off of the adhesive sheet. Suitable release liners are commercially available from, for example, Rexam Release, of Bedford Park, Ill.

The adhesive sheet employed in embodiments of the present invention can be any suitable thickness. However, a typical non-limiting thickness range is from 0.0005 inches to 0.040 inches (excluding the thickness of the light emitter and light detector that are attached to the adhesive sheet). A major surface of the adhesive fluorescence measurement patch (i.e., the surface facing a user's body when the adhesive fluorescence measurement patch is adhered) can have any suitable surface area with a typical surface area being, for example, in the range of from 0.40 square inches to 4 square inches. However, larger surface areas, for example, 40 square inches, can be employed if desired.

Any suitable light emitter 2104 and suitable light detector 2106 known to one skilled in the art can be employed in adhesive fluorescence measurement patches according to embodiments of the present invention. Suitable light emitters can be, for example, light emitting diodes (e.g., light emitting diodes commercially available from Lite-On Technology Corporation of Milpitas, Calif.). Suitable light detectors can be, for example, photodiodes (e.g., photodiodes commercially available from Hamamatsu Corporation of Bridgewater, N.J.).

In FIG. 13A, adhesive fluorescence measurement patch 2100 is depicted as in communication with remote module 2200 via radio frequency signals RF. However, once apprised of the present disclosure, one skilled in the art will recognize that other suitable technologies of providing wireless communication between an adhesive fluorescence measurement patch and a remote module can be employed. A passive RFID tag is employed to store and transmit information, using a non-radiative back-scattering communications mode, and the term “transceiver” as used with reference to this embodiment should be understood accordingly

Remote module 2200 can have any suitable capabilities, including the capability to control of light emitter 2104 and light detector 2106 and the capability to process communications received from adhesive fluorescence measurement patch 2100. For example, remote module 2200 can have the capability to continuously or intermittently correlate fluorescent light detected by light detector 2106 to analyte concentration and to then employ the correlation to control other devices, such as an insulin infusion pump supplying insulin to the patient's abdominal adipose tissue. The infusion pump thus supplies insulin in an amount that depends upon the level of the analyte in the fluid as determined by the remote module 200. Suitable remote controllers, as can be modified by one skilled in the art for use in embodiments of the present invention, are described in international paten application WO 03/071930, which is hereby fully incorporated by reference.

Fluorescent measurement patch 2100 is configured to receive and store measurement data from the light detector 2106, Fluorescent measurement patch 2100 may be configured to convert a measurement signal from light detector 2106 into an ISF or blood glucose concentration value. Alternatively, the conversion might be done by the remote module 2200. Information stored in fluorescent measurement patch 2100 is preferably stored in a passive RFID tag contained within the fluorescent measurement patch; however, it may be otherwise stored so long as the fluorescent measurement patch 2100 has some active or passive RF communications capability by means of which it can communicate with the remote module 2200 when interrogated by the remote module 2200. One form of passive communication is for the fluorescent measurement patch 2100 to extract energy from an RF interrogation signal from the remote controller module 2200 to use in the transmission of the information. It may use the same technique to extract energy from an RF testing signal from the remote controller module 200 to use in the operation of the light emitter 2104 and light detector 2106. The testing and interrogation signals may be the same signal, in which case there may be no need to store the data in the RFID tag at all. Another form of passive communication is for the fluorescent measurement patch 2100 to modulate or modify the interrogation signal, as with a passive RFID tag.

Fluorescent measurement patch 100 may of course be used with a parental or other care-giver monitoring module as discussed above.

Remote module 2200 may be, or maybe incorporated into, a hand-held device, a portable device, a PDA, a mobile telephone, or a laptop computer.

RFID tags in any embodiments of this invention may hold and convey information representing the calibration quantity of the sensors used. The module in which the analyte, e.g. glucose, measurement values are calculated will wirelessly receive that information and use it when determining the level of the analyte in the fluid.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent applications cited in this specification are herein incorporated by reference in their entirety as if each individual publication or patent application were specifically and individually put forth herein.

1-48. (canceled)

49. A system for determining the level of an analyte in a physiological fluid of a user, comprising:
a sensor configured to be attached to a skin of the user to obtain data representative of a level of at least an analyte in the fluid of the user;
an RFID tag in communication with the sensor to receive the data; and
a remote controller configured to interrogate the RFID tag and receive the data from the RFID tag.

50. The system according to claim 49 in which the data directly represents the level of the analyte in the fluid.

51. The system according to claim 49 in which:
the data indirectly represents the level of the analyte in the fluid; and
the receiver is configured to determine, from the data, the level of the analyte in the fluid.

52. The system according to claim 49 in which the fluid comprises the user's blood.

53. The system according to claim 50 in which the physiological fluid is the user's interstitial fluid.

54. The system according to claim 50 in which the level of the analyte is diagnostic of a medical disorder or diseased condition.

55. The system according to claim 54 further comprising a drug dispensing unit configured to dispense a drug to alleviate the medical disorder or disease condition.

56. The system according to claim 55 in which the drug dispensing unit is configured to dispense the drug in an amount that depends upon the level of the analyte in the fluid.

57. The system according to claim 50 in which the analyte comprises an analyte selected from a group consisting essentially of glucose, HbA1C, lactate, cholesterol alcohol, a ketone, urate, a therapeutic drug, a recreational drug, a performance-enhancing drug, a biomarker indicative of a diseased condition, a hormone, an antibody, a metabolite of any of the aforesaid, or combinations thereof.

58. The system according to claim 57 in which the analyte is glucose.

59. The system according to claim 56 in which the analyte is glucose and the drug promotes cellular uptake of glucose.

60. The system according to claim 59 in which the drug comprises insulin or an insulin analog.

61. The system according to claim 60 in which the dispensing unit comprises an infusion pump configured to dispense the drug directly into the body of the user.

62. The system according to claim 61 in which the receiver comprises a local device carried by the user.

63. The system according to claim 60 in which the receiver comprises a device remote from the user.

64. The system according to claim 62 further comprising a device remote from the user in which the local and remote devices are in wireless communication with one another and adapted to transfer from the local device to the remote device either the data received from the sensor or the level of the analyte as determined by the receiver or both.

65. The system according to claim 64 in which:
the remote device is configured to establish a communications link with a public switched telephone network or another circuit-switched communications network, or a mobile telephony network, the internet or another packet-switched communications network; or
the local device is configured to establish a communications link with a mobile telephony network or another wireless communications network.

66. The system according to claim 65 in which the communications link is used to transmit the data received from the sensor or the level of the analyte as determined by the receiver, information concerning the variation of either over time.

67. The system according to claim 66 in which the communications link is used to transmit an alarm condition such as an abnormal analyte level, an abnormal analyte level for a certain time of day, an abnormal analyte level as compared with dietary intake, abnormal or non-functional wireless transfer of information from the sensor or the local device, abnormal physiological fluid sampling frequency, abnormal establishment, or non-establishment, of wireless communication from the sensor or the local device, abnormal storage of information in one of the sensor or local device or other alarm conditions.

68. The system according to claim 50 in which:
the receiver is configured to interrogate the sensor by issuing a wireless interrogation signal; and
the RFID tag is configured to extract energy from the wireless interrogation signal and use the energy extracted to convey data wirelessly to the receiver.

69. The system according to claim 49, in which:
the receiver is configured to interrogate the RFID tag by issuing a wireless interrogation signal;
the RFID tag is configured to modulate or otherwise modify the wireless interrogation signal using the data; and
the receiver is configured to receive back the modulated or otherwise modified interrogation signal and to extract the data from it.

70. The system according to claim 69 in which the interrogation signal is back-scattered by the RFID tag.

71. The system according to claim 49 in which:
the receiver is configured to issue a wireless test signal; and
the RFID tag is configured to extract energy from the wireless test signal and use the energy extracted to obtain the data to be conveyed.

72. The system according to claim 51 in which:
a portion of the sensor, when exposed to the physiological fluid, develops a measurable characteristic that is a function of the level of the analyte in the fluid and of a calibration quantity of the sensor;
the RFID tag is configured to hold and convey information representing the calibration quantity of the sensor; and
the receiver is configured to receive the information representing the calibration quantity of the RFID tag and to use it when determining the level of the analyte in the fluid.

73. A sensor for use in determining the level of an analyte in a physiological fluid of a user, the sensor comprising:
a test module having a portion attached to the skin of the user to sample the fluid to determined an analyte level in the fluid; and
an RFID tag configured to convey data representing the analyte level when wirelessly interrogated by a receiver.

74. The sensor according to claim 73 in which:
at least a portion of the test module, when exposed to the physiological fluid, develops a measurable characteristic that is a function of the level of the analyte in the fluid and of a calibration quantity of the test module; and
the RFID tag is configured to hold and convey information representing a calibration quantity specific to the test module.

75. The sensor according to claim 73 comprising at least one of a photometric or calorimetric sensor.

76. The sensor according to claim 75 in which the sensor comprises:
an intracorporeal part configured to be exposed to the physiological fluid by implantation in the user concerned and, when so exposed, develops a measurable characteristic, being an indicator of the extent to which exposure of the sensor to the fluid affects its optical characteristics, which is a function of the level of the analyte in the fluid; and
an extracorporeal part configured to acquire the measurable characteristic of the intracorporeal part by transdermal wireless communication, and includes the RFID tag configured to convey wirelessly the data representative of the level of the analyte when wirelessly interrogated.

77. The sensor according to claim 76 in which the transdermal wireless communication comprises transdermal optical transmission.

78. The sensor according to claim 77 in which the intracorporeal part comprises a reagent that reversibly binds to the analyte.

79. The sensor according to claim 78 in which the reagent is fluorescent and the measurable characteristic is selected from a group consisting essentially of:
a fluorescence intensity; an emission or excitation spectrum, peak, gradient or ratio; any one or more parts of such a spectrum; an emission polarization; an excited state lifetime; a quenching of fluorescence; a change over time of any of the aforesaid; or any combination thereof.

80. The sensor according to claim 79, in which:
the reagent comprises a donor molecule and an acceptor molecule; and
the measurable characteristic comprises an indicator of the extent to which non-radiative fluorescence resonance energy transfer occurs between the donor and the acceptor upon reversible binding of the reagent to the analyte.

81. The sensor according to claim 79, in which the reagent comprises a specific binding pair, one of which is the donor molecule and the other of which the acceptor molecule.

82. The sensor according to claim 81 in which the sensor comprises a envelope that contains and is substantially impermeable to the reagent, but is permeable to the analyte.

83. The sensor according to claim 82 in which the envelope comprises a microdialysis vessel or a microcapsule or an alginate bead optionally covered with a polylysine covering.

84. The sensor according to claim 83, in which the reagent includes a catalyst and a dye or dye precursor and the catalyst catalyses, in the presence of the analyte, the denaturing of the dye or the conversion of the dye precursor into a dye.

85. The sensor according to claim 84, in which the analyte comprises an analyte selected from a group consisting essentially of glucose, HbA1C, lactate, cholesterol, alcohol, a ketone, urate, a therapeutic drug, a recreational drug, a performance-enhancing drug, a biomarker indicative of a diseased condition, a hormone, an antibody, a metabolite of any of the aforesaid, or combinations thereof.

86. The sensor according to claim 84, in which the analyte comprises glucose, the catalyst comprises a combination of glucose oxidase and horseradish peroxidase and the reagent includes a leuco-dye.

87. The sensor according to claim 86 in which the leuco-dye comprises 2,2-azino-di-[3-ethylbenzthiazoline-sulfonate], tetramethylbenzidine-hydrochloride or 3-methyl-2-benzothiazoline-hydrazone in conjunction with 3-dimethylamino-benzoicacide.

88. The sensor according to claim 64 in which the measurable characteristic is selected from a group consisting essentially of an inter-electrode impedance; an inter-electrode current; a potential difference; an amount of charge; a change over time of any of the aforesaid; and combinations thereof.

89. The sensor according to claim 88, in which the electrochemical sensor comprises a substrate, an electrode layer containing the electrodes, and at least a first reagent layer.

90. The sensor according to claim 89 in which the analyte comprises glucose and the reagent layer comprises glucose oxidase.

91. The system according to claim 50, in which the receiver is, or is incorporated into, a hand-held device, a portable device, a PDA, a mobile telephone, or a laptop computer.

92. The system according to claim 49, in which the RFID tag is configured to store the data pending later interrogation.

93. The sensor according to claim 73, in which the RFID tag is configured to store the data pending later interrogation.

94. A method of transferring data between a sensor attached to a skin surface of a user and a remote receiver, the method comprising:
attaching a sensor to a skin of a user;
testing a fluid sample of the user;
transferring data representing the level of analyte in the fluid sample to a RFID tag; and
transmitting the data from the RFID tag to the remote receiver upon a query by the receiver.

95. The method of claim 94, in which the transmitting comprises inducing power to the RFID tag during the query by the remote receiver.

96. The method of claim 95, in which the inducing comprises modulating signals sent by the remote receiver to include the data by back-scatter of the signals sent by the remote receiver.

97. The method of claim 94, further comprising transferring calibration data specific to the sensor to the remote receiver.
